(19) United States
(12) Patent Application Publication
(10) Pub. No.: US 2010/0305159 A1
(43) Pub. Date: Dec. 2, 2010

(54) CRYSTALLINE FORM OF PIPERIDINE COMPOUND

(75) Inventors: Ryo Kobayashi, Osaka (JP); Satoru Kuraoka, Osaka (JP)

Correspondence Address: BROWDY AND NEIMARK, P.L.L.C.;624 NINTH STREET, NW, SUITE 300, WASHINGTON, DC, 20001-5303, US

(73) Assignee: MITSUBISHI TANABE PHARMA CORPORATION, Osaka-shi, Osaka (JP)

(21) Appl. No.: 12/745,661

(22) PCT Filed: Nov. 28, 2008
(86) PCT No.: PCT/JP2008/072102

§ 371 Date: Jun. 1, 2010

Related U.S. Application Data
(60) Provisional application No. 60/991,019, filed on Nov. 29, 2007.

(30) Foreign Application Priority Data
Nov. 30, 2007 (JP) 2007-309622

(51) Int. Cl. A61K 31/451; C07D 211/46; A61P 29/00; A61P 37/08; A61P 25/06; A61P 11/14; A61P 37/00; A61P 1/08; A61P 13/00; A61P 25/00; A61P 1/00

(52) U.S. Cl. 514/327; 546/222

(57) ABSTRACT

The present invention is to provide a crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine or a solvate thereof.

### TECHNICAL FIELD

The present invention relates to a crystalline form of (2R,4S)-1-{N-(3,5-bistrifluoromethylbenzyl)-N-methyl}-aminocarbonyl-2-(4-fluoro-2-methylphenyl)-4-(2-hydroxy-ethylaminocarbonyloxy)piperidine or a solvate thereof, a process for preparing the same and a use thereof.

### BACKGROUND ART

Tachykinin is a general name for a group of neuro-peptides, and there have been known substance P (herein-after referred to as SP), neurokinin-A, and neurokinin-B in mammals. These peptides are known to exhibit various kinds of biological activities by binding to their corresponding receptors which exist in vivo (neurokinin-1, neurokinin-2, neurokinin-3). Among them, SP is one of those which have the longest history in the neuropeptides, and have been studied in detail. Its existence was confirmed in an extract of horse intestinal tube in 1931, and it was a peptide comprising 11 amino acids, whose structure was determined in 1971.

SP exists widely in central and peripheral nervous systems, and it has physiological activities such as vasodilatation action, vascular permeability promoting action, smooth muscle contracting action, hypertarachia (neuronal excitement) action, salivation action, diuretic action, immunological action, etc., as well as a function of neurotransmitter of the primary sensory neuron. Especially, it is known that SP released from the terminal of posterior horn of spinal cord upon pain impulse transfers pain information to the secondary sensory neuron, and that SP released from the peripheral terminal induces an inflammatory response via its receptors. From these facts, SP is considered to be involved in various diseases (for example, pain, inflammation, allergy, pollakiurea, urinary incontinence, respiratory disease, mental disorder, depression, uneasiness, emesis, etc.), and also, SP is considered to be involved in Alzheimer-type dementia [Review: Physiological Reviews, vol. 73, pp. 229-308 (1993) (Non-Patent Literature 1), Journal of Autonomic Pharmacology, vol. 13, pp. 23-93 (1993) (Non-Patent Literature 2)].

In Patent Literature 1, there is disclosed a piperidine compound which has neurokinin-1 receptor antagonistic action, and is useful as a prophylaxis and treatment agent for central nervous system diseases such as emesis, depression, etc., or urinary disorder such as pollakiurea, etc. Also, in Patent Literature 1, (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine represented by the following structural formula (I):

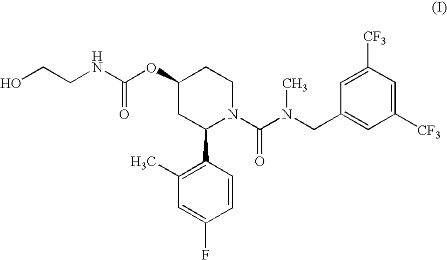

has been described, but the present invention dose not disclose paticular solvates nor polymorphism.

Moreover, when a compound is used as a medical product, a crystalline form is generally preferred in the point of stability thereof, and control of crystalline polymerphism is required in some cases for handling the material or robustness in quality of the same.

### [Non-Patent Literature 1] Physiological Reviews, Vol. 73, pp. 229-308 (1993).

### [Non-Patent Literature 2] Journal of Autonomic Pharmacology, Vol. 13, pp. 23-93 (1993).

### [Patent Literature 1] WO 2003/099787 A

### DISCLOSURE OF THE INVENTION

### Problems to be Solved by the Invention

In order to ensure uniform quality and a certain degree of action and effects required as a medical product which has an excellent tachykinin receptor antagonistic action, and clinical effect of which is sufficiently satisfied as a therapeutic agent of said diseases, it is required that a compound to be used as a medical product be substantially pure crystalline form.

### Means to Solve the Problems

The present invention relates to a crystalline form of (2R,4S)-1-{N-(3,5-bistrifluoromethylbenzyl)-N-methyl}-aminocarbonyl-2-(4-fluoro-2-methylphenyl)-4-(2-hydroxy-ethylaminocarbonyloxy)piperidine or a solvate thereof.

That is, the characteristic features of the present invention relate to

[1] a crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarbonyloxy)piperidine or a solvate thereof,
[2] the crystalline form described in the above-mentioned
[1], wherein the crystalline form is substantially pure polymorphism of Type I,
[3] the crystalline form described in the above-mentioned
[1], wherein the crystalline form has an X-ray powder diffraction pattern having substantially the same as that shown in FIG. 1,
[4] the crystalline form described in the above-mentioned
[1] or the above-mentioned [3], wherein the crystalline form has an IR spectrum having substantially the same as that shown in FIG. 7,
[5] the crystalline form described in the above-mentioned
[1], wherein the crystalline form is substantially pure polymorphism of Type II,
[6] the crystalline form described in the above-mentioned
[1], wherein the crystalline form has an X-ray powder diffraction pattern having substantially the same as that shown in FIG. 2,
[7] the crystalline form described in the above-mentioned
[1] or the above-mentioned [6], wherein the crystalline form has an IR spectrum having substantially the same as that shown in FIG. 8,
[8] the crystalline form described in the above-mentioned
[1], wherein the crystalline form is substantially pure polymorphism of Type VII,
[9] the crystalline form described in the above-mentioned
[1], wherein the crystalline form has an X-ray powder diffraction pattern having substantially the same as that shown in FIG. 6,
[10] the crystalline form described in the above-mentioned
[1] or the above-mentioned [9], wherein the crystalline form has an IR spectrum having substantially the same as that shown in FIG. 11,
[11] the crystalline form described in the above-mentioned
[1], wherein the crystalline form is substantially pure polymorphism of Type III,
[12] the crystalline form described in the above-mentioned
[1], wherein the crystalline form has an X-ray powder diffraction pattern having substantially the same as that shown in FIG. 3,
[13] the crystalline form described in the above-mentioned
[1] or the above-mentioned [12], wherein the crystalline form has an IR spectrum having substantially the same as that shown in FIG. 9,
[14] a process for preparing Type II crystalline form described in the above-mentioned [1], which comprises crystallizing a solution of crystalline form (Type III crystalline form) of (2R,4S)-1-{N-(3,5-bistrifluoromethyl-benzyl)-N-methyl}aminocarbonyl-2-(4-fluoro-2-methylphenyl)-4-(2-hydroxyethylaminocarbonyloxy)piperidine isopropyl alcoholate in the presence of or in the absence of a small amount of Type II crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyl-oxy)piperidine,
[15] a process for preparing Type II crystalline form described in the above-mentioned [1], which comprises crystallizing a solution of amorphous (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)-piperidine in the presence of or in the absence of Type II crystalline form of (2R,4S)-1-{N-(3,5-bistrifluoromethyl-benzyl)-N-methyl}aminocarbonyl-2-(4-fluoro-2-methylphenyl)-4-(2-hydroxyethylaminocarbonyloxy)piperidine.
[16] a medical composition comprising a therapeutically effective amount of the crystalline form described in any one of the above-mentioned [1] to [10], and a pharmaceutically acceptable carrier,
[17] a crystalline form described in any one of the above-mentioned
[1] to [10] for the use of an effective ingredient for therapy,
[18] use of the crystalline form described in any one of the above-mentioned [1] to [10] for the manufacture of a medicine for prophylaxis or treatment of diseases selected from inflammation, allergic diseases, pain, migraine, neuralgia, itchiness, cough, central nervous system diseases, digestive organs disease, nausea, emesis, urinary disorder, circulatory disease and immune disorder,
[19] a method for treating and preventing a disease selected from inflammation, allergic diseases, pain, migraine, neuralgia, itchiness, cough, central nervous system diseases, digestive organs disease, nausea, emesis, urinary disorder, circulatory disease and immune disorder, which comprises administering the crystalline form described in any one of the above-mentioned [1] to [10] in a clinically effective dose to mammal, and
[20] the method described in the above-mentioned [19], wherein the disease is central nervous system diseases, emesis or urinary disorder.

### BRIEF DESCRIPTION OF THE DRAWINGS

FIG. 1 is an X-ray powder diffraction pattern of Type I crystalline form of (2R,4S)-1-{N-(3,5-bistrifluoromethyl-benzyl)-N-methyl}aminocarbonyl-2-(4-fluoro-2-methylphenyl)-4-(2-hydroxyethylaminocarboxyloxy)piperidine. In the figure, the vertical axis represents an intensity (cps), and the horizontal axis represents diffraction angle (2θ: °).

FIG. 2 is an X-ray powder diffraction pattern of Type II crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarboxyloxy)piperidine. In the figure, the vertical axis represents an intensity (cps), and the horizontal axis represents diffraction angle (2θ: °).

FIG. 3 is an X-ray powder diffraction pattern of Type III crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarboxyloxy)piperidine. In the figure, the vertical axis represents an intensity (cps), and the horizontal axis represents diffraction angle (2θ: °).

FIG. 4 is an X-ray powder diffraction pattern of Type IV crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarboxyloxy)piperidine. In the figure, the vertical axis represents an intensity (cps), and the horizontal axis represents diffraction angle (20:0).

FIG. 5 is an X-ray powder diffraction pattern of Type V crystalline form of (2R,4S)-1-{N-(3,5-bistrifluoromethyl-benzyl)-N-methyl}aminocarbonyl-2-(4-fluoro-2-methylphenyl)-4-(2-hydroxyethylaminocarboxyloxy)piperidine. In the figure, the vertical axis represents an intensity (cps), and the horizontal axis represents diffraction angle (2θ: °)

FIG. 6 is an X-ray powder diffraction pattern of Type VII crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarboxyloxy)piperidine. In the figure, the vertical axis represents an intensity (cps), and the horizontal axis represents diffraction angle (2θ: °).

FIG. 7 is an IR infrared absorption spectrum pattern of Type I crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarboxyloxy)piperidine. In the figure, the vertical axis represents a transmittance (%), and the horizontal axis represents an absorption wave number (cm⁻¹).

FIG. 8 is an IR infrared absorption spectrum pattern of Type II crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarboxyloxy)piperidine. In the figure, the vertical axis represents a transmittance (%), and the horizontal axis represents an absorption wave number (cm⁻¹).

FIG. 9 is an IR infrared absorption spectrum pattern of Type III crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarboxyloxy)piperidine. In the figure, the vertical axis represents a transmittance (%), and the horizontal axis represents an absorption wave number (cm⁻¹).

FIG. 10 is an IR infrared absorption spectrum pattern of Type IV crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarboxyloxy)piperidine. In the figure, the vertical axis represents a transmittance (%), and the horizontal axis represents an absorption wave number (cm⁻¹).

FIG. 11 is an IR infrared absorption spectrum pattern of Type VII crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarboxyloxy)piperidine. In the figure, the vertical axis represents a transmittance (%), and the horizontal axis represents an absorption wave number (cm⁻¹).

### EFFECTS OF THE INVENTION

The present invention is to provide a crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine or a solvate thereof, which has an excellent tachykinin receptor antagonistic action, and has sufficiently satisfying clinical effects in the points of safety and sustainability (metabolism, dynamics in vivo and absorption), etc.

### BEST MODE TO CARRY OUT THE INVENTION

An example of the solvate of the present invention is a solvate with an organic solvent and/or water. An example of the organic solvent is an alcohol (methanol, ethanol, isopropanol, butanol, etc.) and/or acetone, etc.

### <Powder X-ray>

Measurement of powder X-ray was carried out by measuring a sample filled in a sample plate according to the conventional manner using MXP3VA manufactured by MAC Science Co., Ltd., Japan according to the following conditions.

### Anticathode: CuKα

### Wavelength: 1.54050 A

Tube current: 35.0 mA
Tube voltage: 40.0 kV
Sampling interval: 0.020 deg
Scanning rate: 4.000 deg/min
Scanning range (2θ): 5° to 40°

Incidentally, in X-ray powder diffraction, a whole pattern is more important on purpose to identify a crystal-line form than intensity, the intensity varies depending on crystal habit, particle size and measurement conditions, and shall not be regulated strictly.

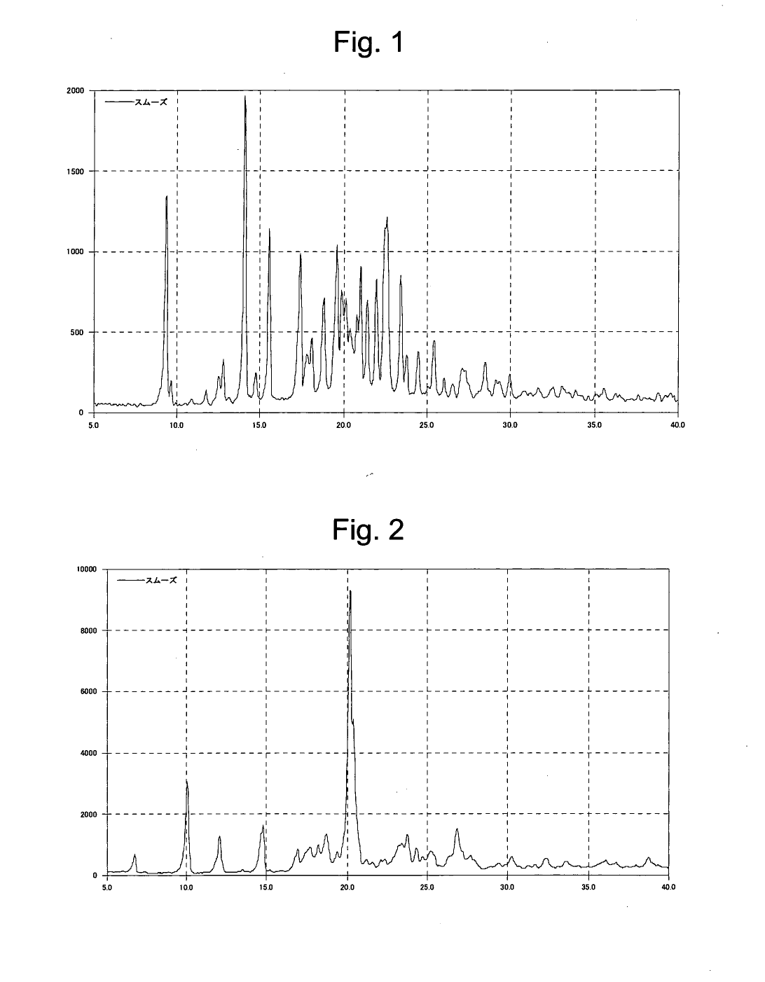

Powder X-ray diffraction patterns of the crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarboxyloxy)piperidine or a solvate thereof according to the present invention are as shown in FIG. 1 to FIG. 6.

### <Infrared Spectrometer>

Measurement of ATR (Attenuated Total Reflection) was carried out by using Spectrum One FT-IR Spectrum Meter manufactured by Perkin-Elmer Inc., U.S.A. as an infrared spectrometer.

Incidentally, to determine a crystallite form of the present compound, powder X-ray is mainly utilized while infrared spectrum is used supplementary.

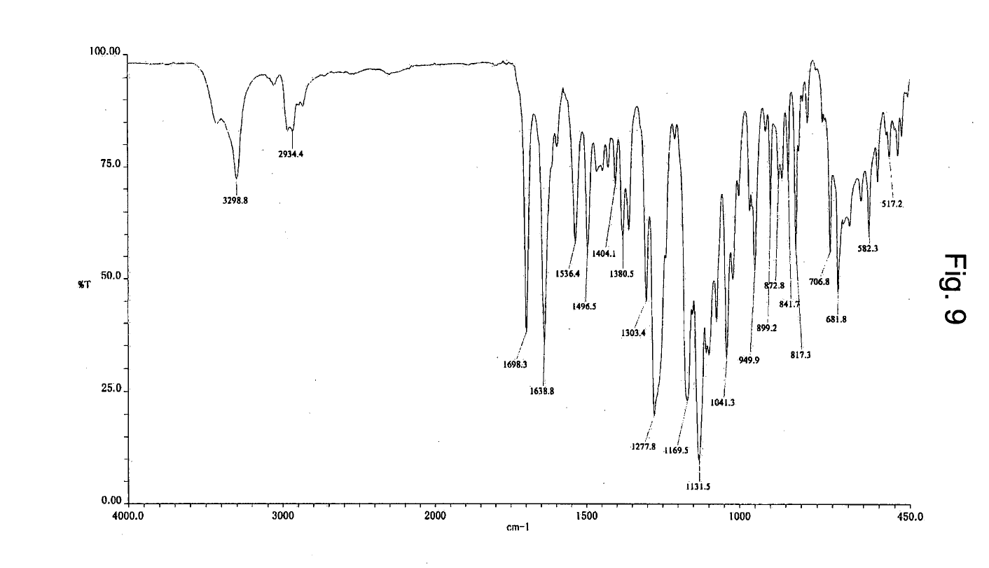

Infrared absorption spectra of the crystalline form of (2R,4S)-1-{N-(3,5-bistrifluoromethylbenzyl)-N-methyl}-aminocarbonyl-2-(4-fluoro-2-methylphenyl)-4-(2-hydroxy-ethylaminocarboxyloxy)piperidine or a solvate thereof according to the present invention are as shown in FIG. 7 to FIG. 11.

### <Thermal Analysis>

Measurements of differential scanning calorimetry (DSC) and thermal gravimetry (TG) were carried out by using SHIMADZU: DSC-50 and TGA-50, respectively, both manufactured by SHIMADZU Corporation, Japan under the following mentioned conditions.

Sample amount: about 5 mg
Sample apparatus: Aluminum pan (Open)
Heating rate: 5° C./min
Measured temperature range: Room temperature to 200 to 300° C.
Atmospheric gas: Dry nitrogen
Atmospheric gas flow amount: 30 ml/min

### <¹H-NMR Measurement>

Measurement of ¹H-NMR (Nuclear Magnetic Resonance) was carried out by using AVANCE400 manufactured by Bruker BioSpin, Germany with deuterated chloroform (CDCl₃).

The crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine or a solvate thereof of the present invention is substantially pure polymorphism of Type I crystalline form, Type II crystalline form, Type III crystalline form, Type IV crystalline form, Type V crystalline form and Type VII crystalline form. Of these, Type I crystalline form, Type II crystalline form and Type VII crystalline form are a crystalline form of (2R,4S)-1-{N-(3,5-bistrifluoromethyl-benzyl)-N-methyl}aminocarbonyl-2-(4-fluoro-2-methylphenyl)-4-(2-hydroxyethylaminocarbonyloxy)piperidine alone (not a crystalline form of a solvate), Type III crystalline form is a crystalline form of isopropyl alcoholate of (2R,4S)-1-{N-(3,5-bistrifluoromethylbenzyl)-N-methyl}aminocarbonyl-2-(4-fluoro-2-methylphenyl)-4-(2-hydroxyethylaminocarbonyl-oxy)piperidine, Type IV crystalline form is a crystalline form of ethanolate of (2R,4S)-1-{N-(3,5-bistrifluorbmethyl-benzyl)-N-methyl}aminocarbonyl-2-(4-fluoro-2-methylphenyl)-4-(2-hydroxyethylaminocarbonyloxy)piperidine, and Type V crystalline form is a crystalline form of acetonate of (2R,4S)-1-{N-(3,5-bistrifluoromethylbenzyl)-N-methyl}amino-carbonyl-2-(4-fluoro-2-methylphenyl)-4-(2-hydroxyethyl-aminocarbonyloxy)piperidine.

Type I crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine of the present invention is a crystalline form showing an X-ray powder diffraction pattern of FIG. 1 when measurement of the powder X-ray is carried out under the above-mentioned conditions. Also, Type I crystalline form of the present invention is a crystalline form showing an infrared absorption spectrum of FIG. 7 when measurement of the infrared absorption spectrum is carried out under the above-mentioned conditions. Furthermore, Type I crystalline form of the present invention is a crystalline form showing a heat absorption peak of a differential scanning calorimetry (DSC) at around 123-127 C when measurement of the DSC is carried out under the above-mentioned conditions.

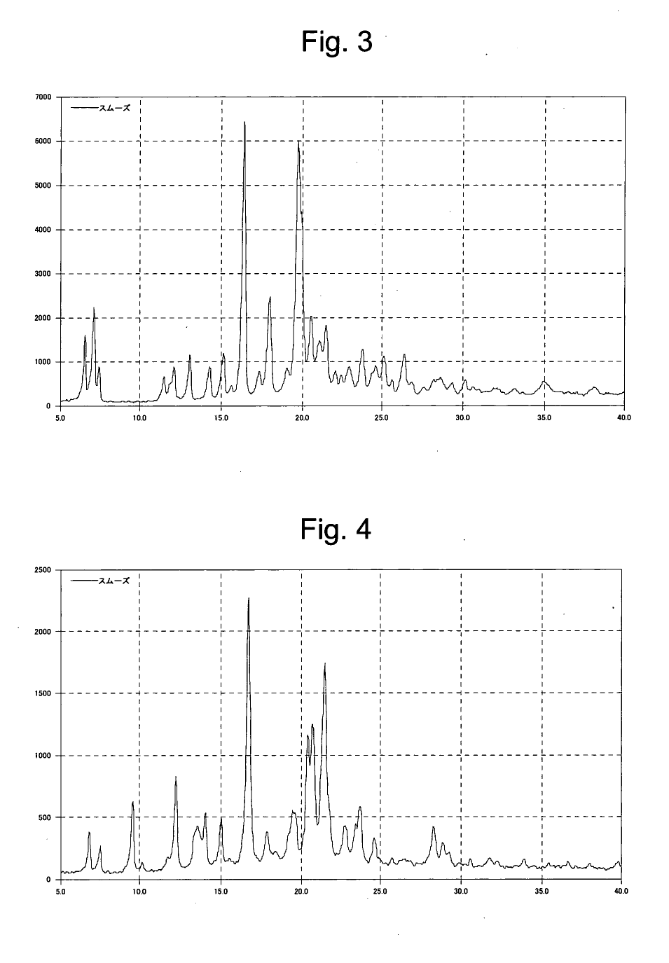

Type II crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine of the present invention is a crystalline form showing an X-ray powder diffraction pattern of FIG. 2 when measurement of the powder X-ray is carried out under the above-mentioned conditions. Also, Type II crystalline form of the present invention is a crystalline form showing an infrared absorption spectrum of FIG. 8 when measurement of the infrared absorption spectrum is carried out under the above-mentioned conditions. Furthermore, Type II crystal-line form of the present invention is a crystalline form showing a heat absorption peak of a differential scanning calorimetry (DSC) at around 106-110° C. when measurement of the DSC is carried out under the above-mentioned conditions.

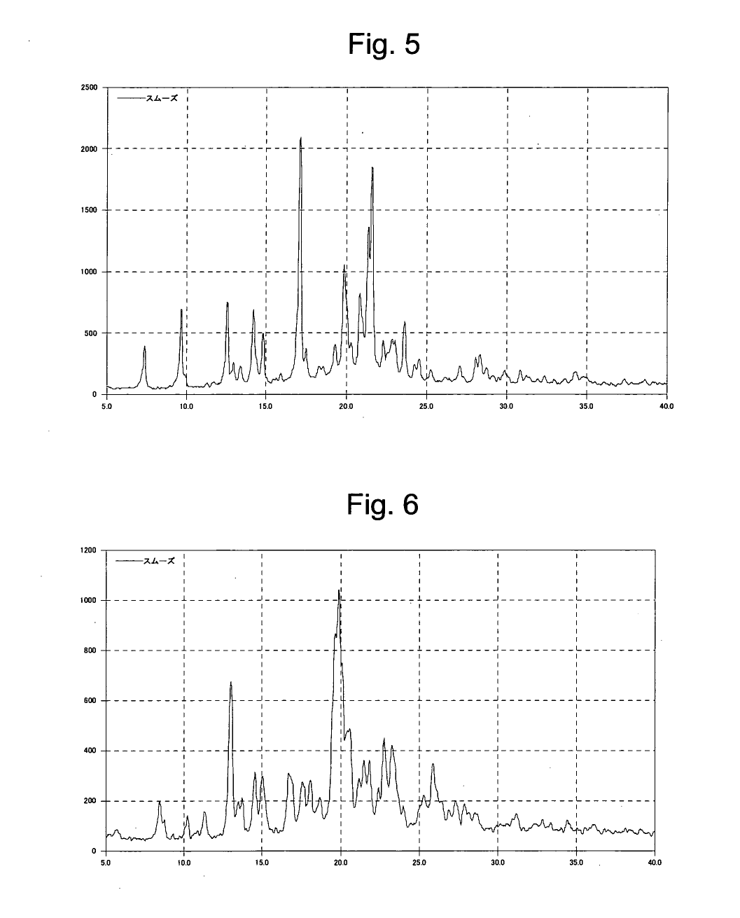

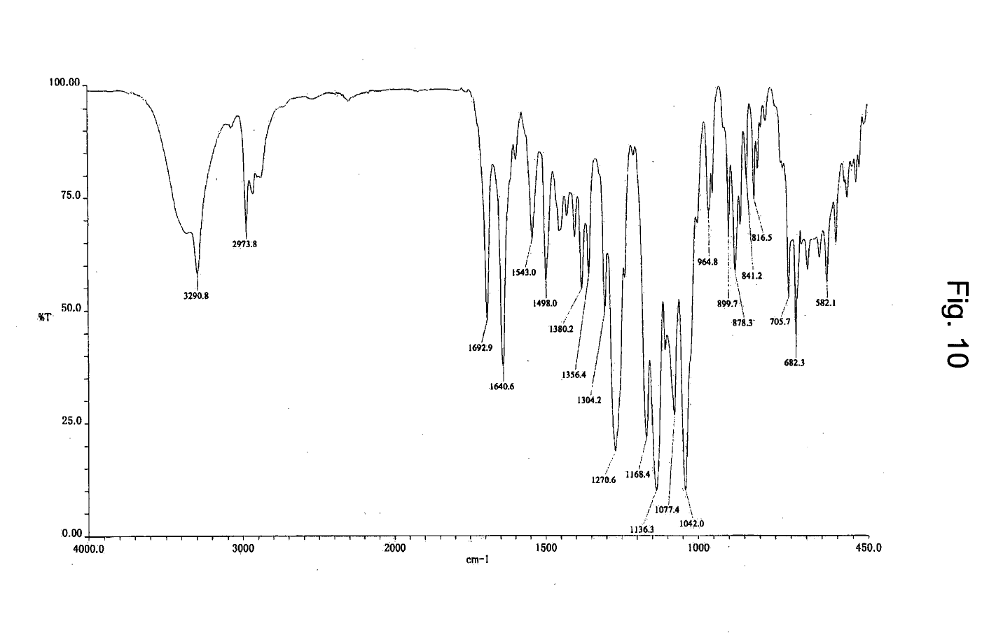

Type III crystalline form of (2R,4S)-1-{N-(3,5-bis-trifluoromethylbenzyl)-N-methyl}aminocarbonyl-2-(4-fluoro-2-methylphenyl)-4-(2-hydroxyethylaminocarbonyloxy)piperidine of the present invention is a crystalline form showing an X-ray powder diffraction pattern of FIG. 3 when measurement of the powder X-ray is carried out under the above-mentioned conditions. Also, Type III crystalline form of the present invention is a crystalline form showing an infrared absorption spectrum of FIG. 9 when measurement of the infrared absorption spectrum is carried out under the above-mentioned conditions. Furthermore, Type III crystalline form of the present invention is a crystalline form showing a heat absorption peak of a differential scanning calorimetry (DSC) at around 76-84° C. when measurement of the DSC is carried out under the above-mentioned conditions.

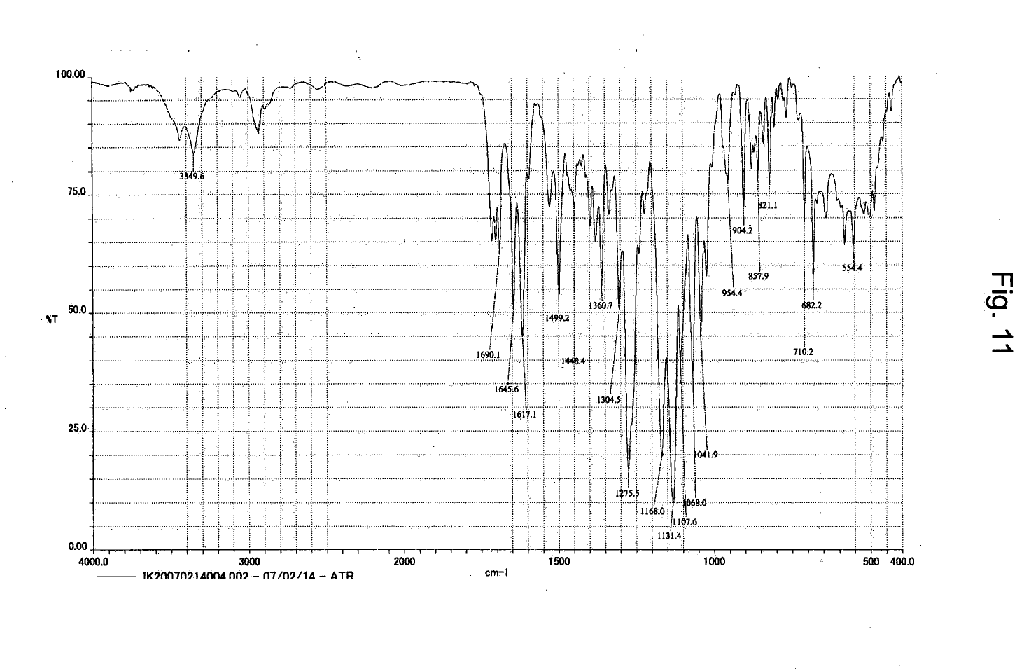

Type IV crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine of the present invention is a crystalline form showing an X-ray powder diffraction pattern of FIG. 4 when measurement of the powder X-ray is carried out under the above-mentioned conditions. Also, Type IV crystalline form of the present invention is a crystalline form showing an infrared absorption spectrum of FIG. 10 when measurement of the infrared absorption spectrum is carried out under the above-mentioned conditions.

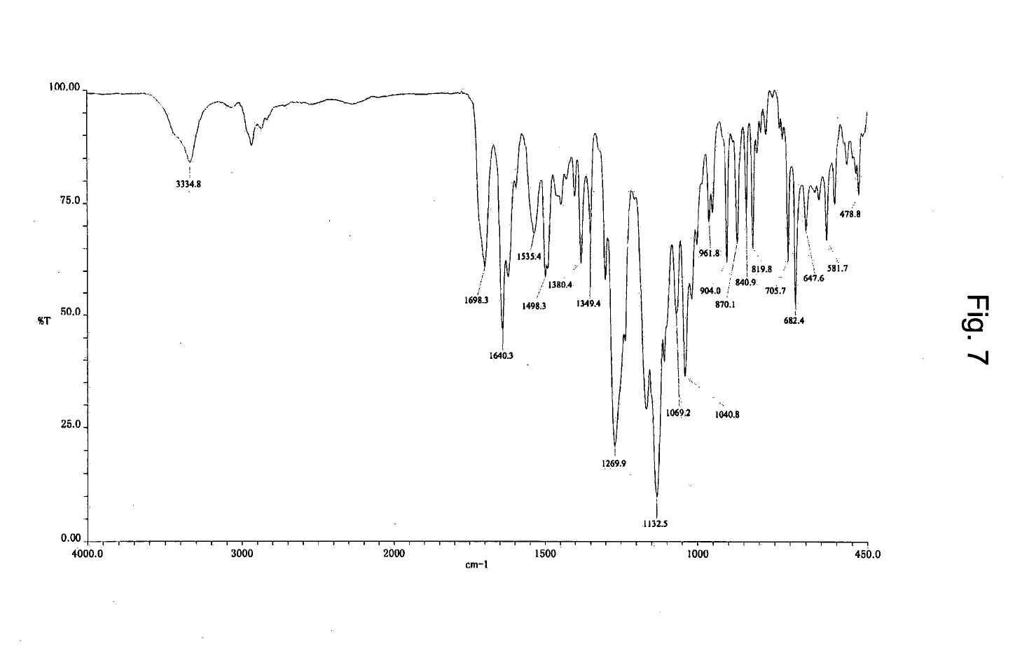

Type V crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine of the present invention is a crystalline form showing an X-ray powder diffraction pattern of FIG. 5 when measurement of the powder X-ray is carried out under the above-mentioned conditions.

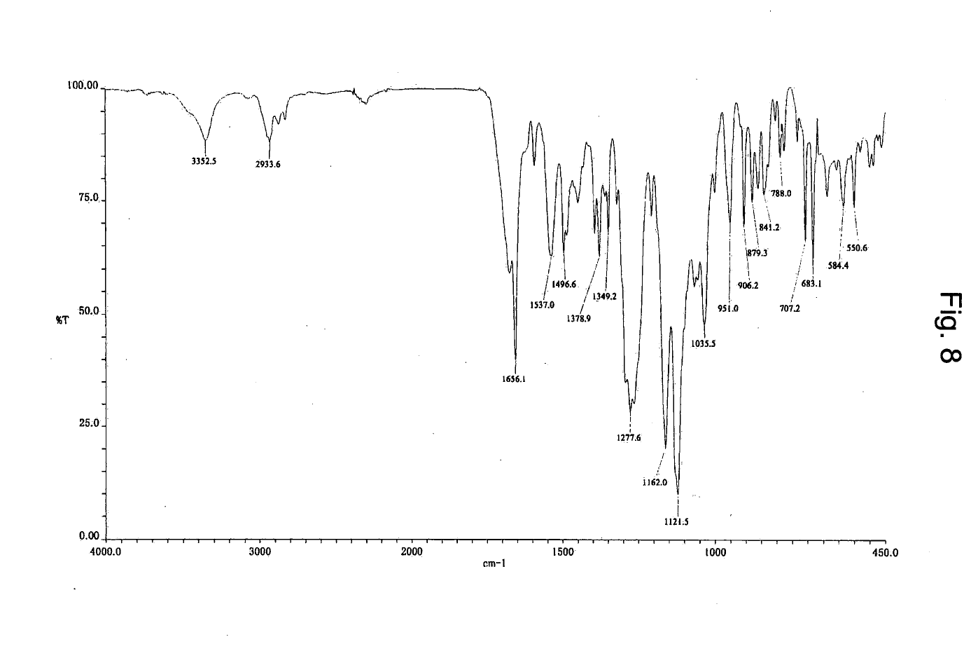

Type VII crystalline form of (2R,4S)-1-{N-(3,5-bis-trifluoromethylbenzyl)-N-methyl}aminocarbonyl-2-(4-fluoro-2-methylphenyl)-4-(2-hydroxyethylaminocarbonyloxy)piperidine of the present invention is a crystalline form showing an X-ray powder diffraction pattern of FIG. 6 when measurement of the powder X-ray is carried out under the above-mentioned conditions. Also, Type VII crystalline form of the present invention is a crystalline form showing an infrared absorption spectrum of FIG. 11 when measurement of the infrared absorption spectrum is carried out under the above-mentioned conditions. Furthermore, Type VII crystalline form of the present invention is a crystalline form showing a heat endothermic peak of a differential scanning calorimetry (DSC) at around 75-79° C. when measurement of the DSC is carried out under the above-mentioned conditions.

The crystalline form of the present invention can be prepared as mentioned below.

Type I crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine of the present invention can be prepared by dissolving amorphous (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine in a solvent such as isopropyl ether, etc., under room temperature, and crystallizing under ice-cooling to 15° C. in the absence of or in the presence of seed crystals of Type I crystalline form.

Type II crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine of the present invention can be prepared by dissolving Type I crystalline form or Type III crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyl-oxy)piperidine or (2R,4S)-1-{N-(3,5-bistrifluoromethyl-benzyl)-N-methyl}aminocarbonyl-2-(4-fluoro-2-methylphenyl)-4-(2-hydroxyethylaminocarbonyloxy)piperidine in a solvent such as toluene, methyl-tert-butyl ether, isopropyl ether, etc., under room temperature to 40° C., adding a solvent such as normal hexane or normal heptane, etc., and crystallizing the same at 5 to 15° C. in the absence of or in the presence of seed crystals of Type II crystalline form.

Type III crystalline form of (2R,4S)-1-{N-(3,5-bis-trifluoromethylbenzyl)-N-methyl}aminocarbonyl-2-(4-fluoro-2-methylphenyl)-4-(2-hydroxyethylaminocarbonyloxy)piperidine of the present invention can be prepared by dissolving Type I crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarbonyloxy)piperidine in a solvent such as isopropyl alcohol, etc., at 45 to 55° C., and crystallizing the same at 10 to 20° C. in the absence of or in the presence of seed crystals of Type III crystalline form.

Type IV crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine of the present invention can be prepared by dissolving Type I crystalline form of amorphous (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine or (2R,4S)-1-{N-(3,5-bistrifluoromethylbenzyl)-N-methyl}-aminocarbonyl-2-(4-fluoro-2-methylphenyl)-4-(2-hydroxy-ethylaminocarbonyloxy)piperidine in a solvent such as ethyl alcohol, etc., at 35 to 45° C., and crystallizing the same at 15 to 25° C. in the absence of or in the presence of seed crystals of Type IV crystalline form.

Type V crystalline form of (2R,4S)-1-{N-(3,5-bistri-fluoromethylbenzyl)-N-methyl}aminocarbonyl-2-(4-fluoro-2-methylphenyl)-4-(2-hydroxyethylaminocarbonyloxy)piperidine of the present invention can be prepared by dissolving Type III crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarbonyloxy)piperidine in a solvent such as acetone, etc., under room temperature, adding a solvent such as water, etc., and crystallizing the same at 5 to 15° C. in the absence of or in the presence of seed crystals of Type V crystalline form.

Type VII crystalline form of (2R,4S)-1-{N-(3,5-bis-trifluoromethylbenzyl)-N-methyl}aminocarbonyl-2-(4-fluoro-2-methylphenyl)-4-(2-hydroxyethylaminocarbonyloxy)piperidine of the present invention can be prepared by dissolving amorphous (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine in a solvent such as isopropyl ether, etc., at 25 to 35° C., crystallizing the same by using seed crystals of Type VII crystalline form, stirring the mixture for a long period of time, and then, adding a solvent such as normal heptane, etc.

In the above-mentioned crystallization, crystal growth is contained.

Some of crystalline forms of the present invention are excellent in the points of stability and hygroscopicity, etc., as compared with those of an amorphous, and either of the Type I, Type II and Type VII crystalline forms of (2R,4S)-1-{N-(3,5-bistrifluoromethylbenzyl)-N-methyl}aminocarbonyl-2-(4-fluoro-2-methylphenyl)-4-(2-hydroxyethylaminocarboxyloxy)piperidine is a non-solvent type, and can be made higher purification by recrystallization so that it is suitable as a starting material for preparing an active pharmaceutical ingredient, and Type II crystalline form is the most suitable of these.

The crystalline form of the present invention has an excellent tachykinin receptor antagonistic action, particularly an SP receptor antagonistic action, whereby it is useful as a safe medicament for prophylaxis and treatment for inflammation or allergic diseases (for example, atopic dermatitis, dermatitis, herpes, psoriasis, asthma, bronchitis, expectoration, rhinitis, rheumatoid arthritis, osteoarthritis, osteoporosis, multiple sclerosis, conjunctivitis, ophthalmia, cystitis, etc.), pain, migraine, neuralgia, itchiness, cough, and further central nervous system diseases [for example, schizophrenia, Parkinson s disease, depression, uneasiness, psychosomatic disorder, morphine dependence, dementia (for example, Alzheimer s disease, etc.), etc.], digestive organs disease [for example, irritable bowel syndrome, ulcerative colitis, Crohn s disease, disorder (for example, gastritis, gastric ulcer, etc.) related to urease-positive Spirillum (for example, helicobacter pylori, etc.), etc.], nausea, emesis, urinary disorder (for example, pollakiurea, urinary incontinence, etc.), circulatory disease (for example, angina pectoris, hypertension, cardiac failure, thrombosis, etc.) and immune disorder, etc., in mammals (for example, mouse, guinea pig, Mongolian gerbil, ferret, rat, hamster, rabbit, cat, dog, bovine, sheep, monkey, human, etc.). Particularly, since the crystalline form of the present invention has a high penetration to the brain and has a low toxicity (high safety), showing almost no side effect, it is useful as a therapeutic or prophylactic agent for central nervous system diseases such as emesis, depression and so forth, or urinary disorder such as pollakiurea, etc. Also, as a mammal, human is preferred.

Measurements on the crystalline form of the present invention can be carried out, for example, according to the method described in European Journal of Pharmacology, vol. 254, pages 221-227 (1994) with respect to a neurokinin-1 receptor binding action, and according to the method described in European Journal of Pharmacology, vol. 265, pages 179-183 (1994) with respect to an action against a neurokinin-1 receptor antagonist inducing action, further according to the method described in Journal of Urology, vol. 155, No. 1, pages 355-360 (1996) with regard to an inhibitory action on pollakiurea: For example, Type II crystalline form of (2R,4S)-1-{N-(3,5-bistrifluoromethyl-benzyl)-N-methyl}aminocarbonyl-2-(4-fluoro-2-methylphenyl)-4-(2-hydroxyethylaminocarboxyloxy)piperidine of the present invention showed a human neurokinin-1 receptor binding action with concentration-dependently.

The crystalline form of the present invention can be administered orally or parenterally, and it can be formulated into a suitable preparation, using a conventionally used pharmaceutically acceptable carrier for an oral or parenteral administration. As such a pharmaceutically acceptable carrier, there may be mentioned, for example, a binder (syrup, Gum Arabic, gelatin, sorbitol, tragacanth, polyvinylpyrrolidone, etc.), an excipient (lactose, sugar, corn starch, potassium phosphate, sorbitol, glycine, etc.), a lubricant (magnesium stearate, talc, polyethylene glycol, silica, etc.), a disintegrator (potato starch, etc.) and a wetting agent (anhydrous lauryl sodium sulfate, etc.), and the like. Also, when these pharmaceutical preparations are administered orally, they may be a solid preparation such as tablets, granules, capsules and powders, or a liquid preparation such as solution, suspension and emulsion. On the other hand, when they are administered parenterally, for example, they can be administered as an injection solution or an infusion solution, using distilled water for injection, physiological saline, aqueous glucose solution, etc., or they may be administered as a suppository, and the like.

An administration dose of the crystalline form of the present invention may vary depending on an administration method, an age, a body weight or conditions of a patient, or a significance of the disease, and, for example, in case of oral administration, it is generally administered in a dose of 0.01 to 20 mg/kg per day, particularly preferably 0.01 to 10 mg/kg per day, and in case of parenteral administration, usually in a dose of 0.01 to 10 mg/kg per day, particularly preferably 0.01 to 1 mg/kg per day.

### EXAMPLES

### Example 1

In 0.50 ml of isopropyl ether was dissolved 100 mg of amorphous (2R,4S)-1-{N-(3,5-bistrifluoromethylbenzyl)-N-methyl}aminocarbonyl-2-(4-fluoro-2-methylphenyl)-4-(2-hydroxyethylaminocarboxyloxy)piperidine at room temperature. The solution was stirred under ice-cooling, and precipitated crystals were filtered to obtain 50 mg of Type I crystalline form wet material of (2R,4S)-1-{N-(3,5-bis-trifluoromethylbenzyl)-N-methyl}aminocarbonyl-2-(4-fluoro-2-methylphenyl)-4-(2-hydroxyethylaminocarboxyloxy)piperidine which shows the following mentioned ¹H-NMR value (δ), and shows an X-ray powder diffraction pattern which appears characteristic peaks of diffraction angles (2θ: °) in X-ray powder diffraction at 9.36, 14.1, 15.56, 17.42, 19.6 and 22.58.

¹H-NMR (CDCl₃) δ:

1.63 (m, 1H), 1.75 (m, 1H), 2.04 (bs, 1H), 2.16 (m, 2H), 2.42 (s, 3H), 2.92 (m, 1H), 2.96 (s, 3H), 3.31 (bs, 2H), 3.43 (m, 1H), 3.69 (m, 2H), 4.36 (m, 2H), 4.65 (m, 1H), 4.80 (m, 1H), 5.00 (bs, 1H), 6.79 (m, 2H), 7.19 (m, 1H), 7.43 (s, 2H), 7.74 (s, 1H)

### Example 2

In 4.9 ml of isopropyl ether was dissolved 1.64 g of amorphous (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine at room temperature, and Type I crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)-piperidine was seeded, and the mixture was stirred at room temperature for 10 minutes. The suspension was cooled to 10° C., precipitated crystals were collected by filtration, and washed with 1.6 ml of cold isopropyl ether. The obtained crystals were vacuum dried at 30° C. for 3 hours to obtain 1.27 g of Type I crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)-piperidine which showed the same characteristic peaks of X-ray powder diffraction pattern as those of Example 1.

### Example 3

In 23.5 ml of isopropyl ether was dissolved 2.35 g of amorphous (2R,4S)-1-{N-(3,5-bistrifluoromethylbenzyl)-N-methyl}aminocarbonyl-2-(4-fluoro-2-methylphenyl)-4-(2-hydroxyethylaminocarboxyloxy)piperidine at 30° C., and Type II crystalline form of (2R,4S)-1-{N-(3,5-bistrifluoromethylbenzyl)-N-methyl}aminocarbonyl-2-(4-fluoro-2-methyl-phenyl)-4-(2-hydroxyethylaminocarboxyloxy)piperidine was seeded, and crystallized. To the suspension was added dropwise to 23.4 ml of normal heptane, the mixture was cooled to 10° C., and the precipitated crystals were collected by filtration, and washed with a cold mixed solvent of 4.7 ml of isopropyl ether/4.7 ml of normal heptane. The obtained crystals were vacuum dried at 30° C. for 2 hours to obtain 1.92 g of Type II crystalline form of (2R,4S)-1-{N-(3,5-bistrifluoromethylbenzyl)-N-methyl}amino-carbonyl-2-(4-fluoro-2-methylphenyl)-4-(2-hydroxyethyl-aminocarboxyloxy)piperidine, which shows the following mentioned ¹H-NMR value (6), and showing an X-ray powder diffraction pattern having characteristic peaks of X-ray powder diffraction with diffraction angles (2θ: °) at 3.38, 10.04, 14.82, 18.74, 20.2, 20.4 and 26.9.

¹H-NMR (CDCl₃) δ:

### 1.63 (m, 1H), 1.75 (m, 1H), 2.04 (bs, 1H), 2.16 (m, 2H), 2.42 (s, 3H), 2.92 (m, 1H), 2.96 (s, 3H), 3.31 (bs, 2H), 3.43 (m, 1H), 3.69 (m, 2H), 4.36 (m, 2H), 4.65 (m, 1H), 4.80 (m, 1H), 5.00 (bs, 1H), 6.79 (m, 2H), 7.19 (m, 1H), 7.43 (s, 2H), 7.74 (s, 1H)

### Example 4

In 2 ml of toluene was dissolved 670 mg of Type I crystalline form of (2R,4S)-1-{N-(3,5-bistrifluoromethyl-benzyl)-N-methyl}aminocarbonyl-2-(4-fluoro-2-methylphenyl)-4-(2-hydroxyethylaminocarboxyloxy)piperidine, and dissolved at room temperature. To the solution was added 2 ml of normal hexane, and the mixture was cooled to 10° C. under stirring to precipitate crystals. To the suspension was added 3.4 ml of normal hexane, the precipitated crystals were collected by filtration, and further washed with 3.4 ml of normal hexane. The obtained crystals were vacuum dried to obtain 480 mg of Type II crystalline form of (2R,4S)-1-{N-(3,5-bistrifluoromethylbenzyl)-N-methyl}amino-carbonyl-2-(4-fluoro-2-methylphenyl)-4-(2-hydroxyethyl-aminocarboxyloxy)piperidine which showed the same characteristic peaks of X-ray powder diffraction pattern as those of Example 3.

### Example 5

In 3.0 ml of methyl tert-butyl ether was dissolved 1.0 g of amorphous (2R,4S)-1-{N-(3,5-bistrifluoromethyl-benzyl)-N-methyl}aminocarbonyl-2-(4-fluoro-2-methylphenyl)-4-(2-hydroxyethylaminocarbonyloxy)piperidine at 30° C., 3.0 ml of normal heptane was added to the solution, then, Type II crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarbonyloxy)piperidine was seeded, and the mixture was stirred at room temperature. To the suspension was added dropwise 6.0 ml of normal heptane, the mixture was cooled to 10° C., and the precipitated crystals were collected by filtration and washed with a mixed solvent of 1 ml of methyl tert-butyl ether and 3 ml of normal heptane. The obtained crystals were vacuum dried at 50° C. for 1.5 hours to obtain 0.74 g of Type II crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine which showed the same characteristic peaks of X-ray powder diffraction pattern as those of Example 3.

### Example 6

0.30 ml of isopropyl alcohol was added to 100 mg of Type I crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarboxyloxy)piperidine, and the crystalline form was dissolved at 50° C. The solution was cooled to 15° C., and the precipitated crystals were collected by filtration to obtain 18 mg of a wet product of (2R,4S)-1-{N-(3,5-bistrifluoromethylbenzyl)-N-methyl}amino-carbonyl-2-(4-fluoro-2-methylphenyl)-4-(2-hydroxyethyl-aminocarboxyloxy)piperidine-isopropyl alcoholate (Type III crystalline form) which showed an X-ray powder diffraction pattern having characteristic peaks of X-ray powder diffraction with diffraction angles (2θ: °) at 7.14, 16.4, 18.0, 19.76, 19.94 and 20.58.

### Example 7

28 ml of isopropyl acetate and 14 ml of water were warmed, then, 2.97 g of (2R,4S)-2-(4-fluoro-2-methyl-phenyl)-4-(2-hydroxyethylaminocarbonyloxy)piperidine, 1.44 g of potassium carbonate and 3.17 g of (3,5-bistrifluoro-methylbenzyl)-N-methyl-aminocarbonyl chloride were successively added to the solution in this order, and the mixture was stirred at 60° C. for 21 hours. To the reaction solution was added 28 ml of toluene, and the liquids were separated. The organic layer was washed with aqueous hydrochloric acid and with water, and the organic layer was concentrated. To the residue was added 12 ml of toluene, and the mixture was concentrated again to remove isopropyl acetate. To the residue were further added 12 ml of isopropyl alcohol and 15 ml of normal heptane, and the mixture was heated to dissolve the residue. The solution was seeded with (2R,4S)-1-{N-(3,5-bistrifluoromethylbenzyl)-N-methyl}amino-carbonyl-2-(4-fluoro-2-methylphenyl)-4-(2-hydroxyethyl-aminocarboxyloxy)piperidine isopropyl alcoholate (Type III crystalline form) at 34° C., and crystal growth was carried out at 30 to 34° C. for 1 hour. Then, 21 ml of normal heptane was added dropwise to the mixture, the mixture was cooled, and the precipitated crystals were collected by filtration at 10° C. The obtained crystals were washed with a mixed solution of 2 ml of isopropyl alcohol and 6 ml of normal heptane, and vacuum dried at 40° C. to obtain 5.14 g of (2R,4S)-1-{N-(3,5-bistrifluoromethylbenzyl)-N-methyl}-aminocarbonyl-2-(4-fluoro-2-methylphenyl)-4-(2-hydroxy-ethylaminocarboxyloxy)piperidine

III crystalline form) which shows the following mentioned

¹H-NMR value (δ), and showing the same characteristic peaks of X-ray powder diffraction pattern as those of Example 6.

¹H-NMR (CDCl₃) δ:

1.63 (m, 1H), 1.75 (m, 1H), 2.04 (bs, 1H), 2.16 (m, 2H), 2.42 (s, 3H), 2.92 (m, 1H), 2.96 (s, 3H), 3.31 (bs, 2H), 3.43 (m, 1H), 3.69 (m, 2H), 4.36 (m, 2H), 4.65 (m, 1H), 4.80 (m, 1H), 5.00 (bs, 1H), 6.79 (m, 2H), 7.19 (m, 1H), 7.43 (s, 2H), 7.74 (s, 1H)

### Example 8

1.0 ml of isopropyl alcohol was added to 333 mg of Type I crystalline form of (2R,4S)-1-{N-(3,5-bistrifluoro-methylbenzyl)-N-methyl}aminocarbonyl-2-(4-fluoro-2-methyl-phenyl)-4-(2-hydroxyethylaminocarboxyloxy)piperidine, and the crystalline form was dissolved by heating. To the solution was seeded (2R,4S)-1-{N-(3,5-bistrifluoromethyl-benzyl)-N-methyl}aminocarbonyl-2-(4-fluoro-2-methylphenyl)-4-(2-hydroxyethylaminocarboxyloxy)piperidine.isopropyl alcoholate (Type III crystalline form) at 40° C. to effect crystallization. To the suspension was added dropwise 2.0 ml of water, the mixture was cooled to 5° C., and the precipitated crystals were collected by filtration and vacuum dried at 40° C. for 3 hours to obtain 324 mg of (2R,4S)-1-{N-(3,5-bistrifluoromethylbenzyl)-N-methyl}amino-carbonyl-2-(4-fluoro-2-methylphenyl)-4-(2-hydroxyethyl-aminocarboxyloxy)piperidine isopropyl alcoholate (Type III crystalline form) which showed the same characteristic peaks of X-ray powder diffraction pattern as those of Example 3.

### Example 9

0.30 ml of ethyl alcohol was added to 100 mg of Type I crystalline form of (2R,4S)-1-{N-(3,5-bistrifluoromethyl-benzyl)-N-methyl}aminocarbonyl-2-(4-fluoro-2-methylphenyl)-4-(2-hydroxyethylaminocarboxyloxy)piperidine, and the crystalline form was dissolved at 35° C. The solution was cooled to 15° C., and the precipitated crystals were collected by filtration to obtain 21 mg of a wet material of (2R,4S)-1-{N-(3,5-bistrifluoromethylbenzyl)-N-methyl}-aminocarbonyl-2-(4-fluoro-2-methylphenyl)-4-(2-hydroxy-ethylaminocarboxyloxy)piperidine ethyl alcoholate product (Type IV crystalline form) which showed an X-ray powder diffraction pattern having characteristic peaks of X-ray powder diffraction with diffraction angles (2θ: °) at 9.56, 12.24, 16.72, 20.42, 20.72, 21.48 and 23.72.

### Example 10

0.2 ml of acetone was added to 200 mg of (2R,4S)-1-{N-(3,5-bistrifluoromethylbenzyl)-N-methyl}aminocarbonyl-2-(4-fluoro-2-methylphenyl)-4-(2-hydroxyethylaminocarboxyl-oxy)piperidine isopropyl alcohol (Type III crystalline form), and the crystalline form was dissolved. To the solution was added dropwise 0.6 ml of water to effect crystallization. The suspension was cooled to 10° C., and the precipitated crystals were collected by filtration and vacuum dried at 40° C. for 2 hours to obtain 146 mg of Type V crystalline form of (2R,4S)-1-{N-(3,5-bistrifluoromethyl-benzyl)-N-methyl}aminocarbonyl-2-(4-fluoro-2-methylphenyl)-4-(2-hydroxyethylaminocarboxyloxy)piperidine. acetonate which showed an X-ray powder diffraction pattern having characteristic peaks of X-ray powder diffraction with diffraction angles (2θ: °) at 12.56, 17.14, 19.86, 20.82, 21.36 and 21.56.

### Example 11

In 23.5 ml of isopropyl ether was dissolved 2.35 g of amorphous (2R,4S)-1-{N-(3,5-bistrifluoromethylbenzyl)-N-methyl}aminocarbonyl-2-(4-fluoro-2-methylphenyl)-4-(2-hydroxyethylaminocarboxyloxy)piperidine at 30° C. To the solution was seeded Type II crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarboxyl-oxy)piperidine to effect crystallization. The suspension was stirred at 10° C. for 66 hours. The precipitated crystals were collected by filtration, washed with a cold mixed solvent of 4.7 ml of isopropyl ether/4.7 ml of normal heptane, and the obtained crystals were vacuum dried at 30° C. for 2 hours to obtain 2.12 g of Type VII crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarboxyloxy)piperidine which shows the following mentioned ¹H-NMR value (8), and showed an X-ray powder diffraction pattern having characteristic peaks of X-ray powder diffraction with diffraction angles (2θ: °) at 12.98, 19.66, 19.88, 20.1, 20.58, 22.76 and 23.26.

¹H-NMR (CDCl₃) δ:

1.63 (m, 1H), 1.75 (m, 1H), 2.04 (bs, 1H), 2.16 (m, 2H), 2.42 (s, 3H), 2.92 (m, 1H), 2.96 (s, 3H), 3.31 (bs, 2H), 3.43 (m, 1H), 3.69 (m, 2H), 4.36 (m, 2H), 4.65 (m, 1H), 4.80 (m, 1H), 5.00 (bs, 1H), 6.79 (m, 2H), 7.19 (m, 1H), 7.43 (s, 2H), 7.74 (s, 1H)

### Example 12

Stabilities of crystalline forms of Type I, Type II and Type VII, and amorphous of (2R,4S)-1-{N-(3,5-bistri-fluoromethylbenzyl)-N-methyl}aminocarbonyl-2-(4-fluoro-2-methylphenyl)-4-(2-hydroxyethylaminocarboxyloxy)piperidine were observed under the conditions of at 40° C./75% RH in a glass bottle with an open state for 4 weeks. As a result, no change in impurity profile of each form of the present invention has been admitted, but in amorphous substance, moisture absorption was admitted and marked change in appearance (white powder→white lump) was admitted. In the crystalline forms of Type I, Type II and Type VII, no change in appearance was observed. Accordingly, improvement in stability according to crystallization could be confirmed.

### Example 13

With regard to Type I and Type II crystalline forms of (2R,4S)-1-{N-(3,5-bistrifluoromethylbenzyl)-N-methyl}-aminocarbonyl-2-(4-fluoro-2-methylphenyl)-4-(2-hydroxy-ethylaminocarboxyloxy)piperidine, hygroscopicity was measured according to the following mentioned conditions. As a result, no change in weight was observed under high humidity conditions so that there was no hygroscopicity.

Device: Dynamic Vapor Sorption device DVS-1 manufactured by Surface Measurement Systems, UK
Amount of specimen: 10 mg
Measured temperature: 25° C.
Relative humidity range: 0 to 90%
Experimental example 1 Neurokinin-1 (NK1) receptor binding inhibitory action Human lymphoblastoma IM-9 cells (5 10⁶ cell/tube) were reacted with 0.3 nM [³H] (Sar⁹, Met¹¹ (O₂)) Substance P and the prepared Type II crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarboxyloxy)-piperidine in 0.5 ml of 50 mM Tris-HCl (pH 7.4, 25° C.) containing 150 mM NaCl, 3 mM MnCl₂, 40 μg/ml bacitracin, 4 μg/ml Leupeptin, 4 μg/ml Chymostatin, 4 μg/ml Phosphoramidon and 0.02% bovine serum albumin at room temperature for 60 minutes according to the method described in European Journal of Pharmacology, vol. 254, pp. 221-227 (1994). The reaction mixture was subjected to suction filtration with a GF/C glass filter which had previously been treated with 0.3% polyethyleneimine, washed with 0.3 ml of ice-cold reaction buffer containing no bovine serum albumin and various kinds of protein decomposition enzyme inhibitors twice, and radioactivity (dpm) on the filter was measured by a liquid scintillation counter. A specific binding amount was obtained by subtracting non-specific binding amount (L-703606 (10 μM) having NK1 receptor antagonistic action) from a total binding amount. As a result, Type II crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarboxyloxy)-piperidine inhibited said Substance P with concentration-dependently, and an IC₅₀ value was 4.59 nM and a Ki value was 2.22 nM.

### Reference Example 1

In 2.0 ml of dichloromethane was dissolved 560 mg of (2R,4S)-1-{N-(3,5-bistrifluoromethylbenzyl)-N-methyl}amino-carbonyl-2-(4-fluoro-2-methylphenyl)-4-hydroxypiperidine, and 1.5 ml of a dichloromethane solution containing 0.22 ml of pyridine and 0.314 ml of phenyl chloroformate was added to the solution under nitrogen atmosphere and under ice-cooling. The reaction solution was stirred at 0° C. to 5° C. for 3.5 hours, and further stirred at room temperature for 16 hours. The reaction solution was poured into water, and dichloromethane was added to the mixture to extract the same twice. The organic layers were combined, washed with 1M hydrochloric acid aqueous solution twice, and further with saturated brine, dried, and then concentrated. The residue was dissolved in 11 ml of N,N-dimethylformamide, 0.274 ml of ethanolamine was added to the mixture under nitrogen atmosphere at room temperature, and the resulting mixture was stirred at 60° C. for 26 hours. The reaction solution was poured into water, and ethyl acetate was added to the mixture to extract twice. The organic layers were combined, washed with saturated brine, dried, and then concentrated. The residue was purified by silica gel column chromatography (chloroform:methanol=39:1) to obtain 493 mg of amorphous (2R,4S)-1-{N-(3,5-bistrifluoromethyl-benzyl)-N-methyl}aminocarbonyl-2-(4-fluoro-2-methylphenyl)-4-(2-hydroxyethylaminocarbonyloxy)piperidine.

MS: 580 [M⁺+1].

### UTILIZABILITY IN INDUSTRY

Crystalline form of the present invention can be used as a starting material for preparing a medicine.

1. A crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine or a solvate thereof.

2. The crystalline form according to claim 1, wherein the crystalline form is substantially pure polymorphism of Type I.

3. The crystalline form according to claim 1, wherein the crystalline form has an X-ray powder diffraction pattern having substantially the same as that shown in FIG. 1.

4. The crystalline form according to claim 1, wherein the crystalline form has an IR spectrum having substantially the same as that shown in FIG. 7.

5. The crystalline form according to claim 1, wherein the crystalline form is substantially pure polymorphism of Type II.

6. The crystalline form according to claim 1, wherein the crystalline form has an X-ray powder diffraction pattern having substantially the same as that shown in FIG. 2.

7. The crystalline form according to claim 1, wherein the crystalline form has an IR spectrum having substantially the same as that shown in FIG. 8.

8. The crystalline form according to claim 1, wherein the crystalline form is substantially pure polymorphism of Type VII.

9. The crystalline form according to claim 1, wherein the crystalline form has an X-ray powder diffraction pattern having substantially the same as that shown in FIG. 6.

10. The crystalline form according to claim 1, wherein the crystalline form has an IR spectrum having substantially the same as that shown in FIG. 11.

11. The crystalline form according to claim 1, wherein the crystalline form is substantially pure polymorphism of Type III.

12. The crystalline form according to claim 1, wherein the crystalline form has an X-ray powder diffraction pattern having substantially the same as that shown in FIG. 3.

13. The crystalline form according to claim 1, wherein the crystalline form has an IR spectrum having substantially the same as that shown in FIG. 9.

14. A process for preparing Type II crystalline form according to claim 1, which comprises crystallizing a solution of crystalline form (Type III crystalline form) of (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine. isopropyl alcoholate in the presence of or in the absence of a small amount of Type II crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine.

15. A process for preparing Type II crystalline form according to claim 1, which comprises crystallizing a solution of amorphous (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine in the presence of or in the absence of Type II crystalline form of (2R,4S)-1-{N-(3,5-bistrifluoromethylbenzyl)-N-methyl}aminocarbonyl-2-(4-fluoro-2-methylphenyl)-4-(2-hydroxyethylaminocarbonyloxy)piperidine.

16. A medical composition comprising a therapeutically effective amount of the crystalline form according to claim 1, and a pharmaceutically acceptable carrier.

17. A crystalline form according to claim 1 for the use of an effective ingredient for therapy.

18. (canceled)

19. A method for treating and preventing a disease selected from inflammation, allergic diseases, pain, migraine, neuralgia, itchiness, cough, central nervous system diseases, digestive organs disease, nausea, emesis, urinary disorder, circulatory disease and immune disorder, which comprises administering the crystalline form according to claim 1 in a clinically effective dose to mammal.

20. The method according to claim 19, wherein the disease is central nervous system diseases, emesis or urinary disorder.
